(19) United States
(12) Patent Application Publication
(10) Pub. No.: US 2015/0290193 A1
(43) Pub. Date: Oct. 15, 2015

(54) NOVEL APPROACHES FOR INDIVIDUALIZED THERAPY OF PANCREATIC DUCTAL ADENOCARCINOMA

(71) Applicant: HI-STEM gGmbH, Heidelberg (DE)

(72) Inventors: Christian Thomas Eisen, Heidelberg (DE); Andreas Trumpp, Heidelberg (DE); Martin Ronald Sprick, Heidelberg (DE)

(21) Appl. No.: 14/434,023

(22) PCT Filed: Oct. 14, 2013
(86) PCT No.: PCT/EP2013/003086

§ 371 Date: Apr. 7, 2015

(30) Foreign Application Priority Data
Oct. 12, 2012 (EP) 12007129.5

(51) Int. Cl. A61K 31/506; A61K 31/7068

(52) U.S. Cl. CPC A61K 31/506; A61K 31/7068

(57) ABSTRACT

This invention relates to novel approaches with respect to the treatment of pancreatic ductal adenocarcinoma (PDAC), in particular to novel PDAC subtype-specific inhibitors, and to methods of treatment utilizing compositions comprising said inhibitors and exploiting PDAC subtype-specific sensitivities to therapeutic agents.

### FIELD OF THE INVENTION

This invention relates to novel approaches with respect to the treatment of pancreatic ductal adenocarcinoma (PDAC), in particular to PDAC subtype-specific inhibitors, and to methods of treatment utilizing compositions comprising said inhibitors and exploiting PDAC subtype-specific sensitivities to therapeutic agents.

### BACKGROUND OF THE INVENTION

Personalized oncology has the potential to revolutionize the way cancer patients will be treated in the future. Different entities of cancer can be divided into subclasses based on molecular differences, including the specific activation of signaling pathways that often determine therapy response and clinical outcome. For various cancer entities including breast, lung and colon cancer, the identification of such subtypes and the possibility to stratify patients into cohorts has already been translated into clinical practice to treat patients in a subtype-specific manner.

PDAC is the most frequent pancreatic cancer and the fourth cause of cancer death in the United States and Europe. Most patients die within 12 months, and only 2% survive five years after prognosis. Little progress in the treatment of PDAC has been made since the approval of Gemcitabine in 2000 and Erlotinib in 2005. Moreover, recent trials with targeted therapies have shown limited or no benefit.

PDAC is still classified as a single cancer entity and is clinically treated as such. However, the existence of three PDAC subtypes termed classical, quasi-mesenchymal and exocrine-like has recently been suggested (Collisson, E. A., Sadanandam, A., Olson, P., Gibb, W. J., Truitt, M., Gu, S. D., Cooc, J., Weinkle, J., Kim, G. E., Jakkula, L., et al. (2011). Subtypes of pancreatic ductal adenocarcinoma and their differing responses to therapy. Nat Med 17, 500-U140). The identification of these subtypes was based on comparative gene expression analysis in micro-dissected epithelial cells form patient specimens.

The existence of PDAC subtypes raises the possibility of inter-subtype specific differences regarding the sensitivity to therapeutic agents. PDAC subtype-specific vulnerabilities might prove crucial for the development of novel individualized therapy approaches of PDAC. The great need for improved methods of treatment of PDAC based on patient stratification has not been met so far.

Previous attempts to predict drug sensitivities in PDAC patients relied on the analysis of gene-expression profiles obtained from patient specimens or the analysis of model cell lines. Both approaches have their limitations with respect to the identification of markers that accurately predict subtype specific drug sensitivities.

The RNA obtained from patient specimens is frequently a mixture of RNA that is derived from the tumor cells with the ones that are derived from the surrounding tumor stroma such as fibroblasts and immune cells. The resulting gene-expression profiles are thus frequently difficult to interpret and might not reflect the expression in the tumor cells.

Cultured cell lines provide a source of stroma-free tumor RNA and thus allow a more sensitive detection of gene-expression profiles and drug sensitivities in that particular cell. The previously available cell lines for PDAC have the limitation that they do not accurately represent the molecular phenotype and expression profiles of the originating tumors. This limitation is especially apparent as it was demonstrated that the available cell lines encompassed only two of the three described PDAC subtypes while a third, the exocrine-like subtype was not represented (Collisson et al., loc. cit.). Thus, it has so far been difficult to predict and test drug sensitivities for all PDAC subtypes, so that the development of PDAC subtype-specific treatment schemes has not yet been possible.

### OBJECTS OF THE INVENTION

It was an object of the invention to provide improved methods of treatment for PDAC exploiting subtype-specific drug sensitivities. In particular, the development of novel methods for individualized therapy of PDAC utilizing compositions comprising PDAC subtype-specific inhibitors is intended by the present invention. Such PDAC subtype-specific inhibitors would satisfy the great need for improved PDAC treatment involving patient stratification into subtype-specific drug treatment cohorts.

### SUMMARY OF THE INVENTION

Surprisingly it has been found that distinct vulnerabilities of the classical PDAC subtype and of certain PDAC of the quasi-mesenchymal subtype can be identified using novel patient-specific models for these PDAC subtypes. These in vitro and in vivo models faithfully recapitulate the human disease and allow for molecular classification and characterization of the classical and a certain part of the quasi-mesenchymal PDAC subtype. Stable PDAC cell lines, which retain tumor-initiating activity in vivo and the gene expression pattern associated with the original tumor subtype, can be used to identify drug sensitivities and targetable pathways by means of Gene-Set Enrichment Analysis (GSEA). Furthermore, it was surprisingly possible for the first time to identify PDAC subtype-specific inhibitors based on the established subtype-specific vulnerabilities. In vitro drug screens using the novel PDAC models revealed striking inter-subtype-specific differences regarding the sensitivity to chemotherapeutics that are commonly used for PDAC as well as to therapeutic agents for the treatment of PDAC demonstrating the need for patient stratification. The newly identified PDAC subtype-specific inhibitors might therefore be useful for the establishment of individualized therapy approaches for greatly improved PDAC treatment.

Thus, in one aspect, the present invention relates to a Src inhibitor for use in the treatment of PDAC of the classical subtype and/or of a Src inhibitor sensitivity predictor-positive PDAC subtype.

In another aspect, the present invention relates to a method for the treatment of PDAC of the classical subtype or of a Src inhibitor sensitivity predictor-positive PDAC subtype comprising the step of administering a Src inhibitor to a patient in need thereof.

In another aspect, the present invention relates to a Src inhibitor for use in the treatment of (a) PDAC of the classical subtype and/or (b) a Src inhibitor sensitivity predictor-positive PDAC subtype, in combination with a second chemotherapeutic agent.

In another aspect, the present invention relates to a method for the combination treatment of (a) PDAC of the classical subtype or (b) a Src inhibitor sensitivity predictor-positive PDAC subtype, comprising the step of administering a Src inhibitor in combination with a second therapeutic agent to a patient in need thereof.

### FIGURES

FIG. 1 shows a flow-chart for the method for predicting Src inhibitor sensitivity in PDAC using immunohistochemical markers: the prediction of Src inhibitor sensitivity in a tumor sample is performed by immunohistochemistry using two markers (method 1).

FIG. 2 shows a flow-chart for the method for predicting Src inhibitor sensitivity in PDAC using a gene signature-based prediction of Src inhibitor sensitivity: the sensitivity is determined in a tumor sample by using gene-expression profiling and the SRC-SP predictor signature (method 2).

FIG. 3 demonstrates the utility of the SRC-SP signature to predict the SRC inhibitor sensitivity from RNA derived from PDAC cell line models. The SRC-SP signature-based method for sensitivity prediction was applied to 12 individual cell lines derived from PDAC patients. The table shows the comparison of the predicted and the experimentally determined SRC inhibitor sensitivity. Cells were classified as SRC inhibitor sensitive if their IC₅₀ for Dasatinib and Sarcatinib was <1 μM when assayed as described in Example 3. Cut-off: FDR 0.2; correct class prediction: 100% (12/12).

FIG. 4 demonstrates the utility of the SRC-SP signature to predict the SRC inhibitor sensitivity from RNA from patient-derived tumor xenografts. The SRC-SP signature-based method for sensitivity prediction was applied to 12 individual xenografts derived from PDAC patients. The table shows the comparison of the predicted and the experimentally determined SRC inhibitor sensitivity. Tumors were classified as SRC inhibitor sensitive if the IC₅₀ for their derived cell lines to Dasatinib and Sarcatinib was <1 μM when assayed as described in Example 3. Cut-off: FDR 0.2; correct class prediction: 83% (10/12).

FIG. 5 shows the IC₅₀ values of PDAC-derived cell lines to Dasatinib and Sarcatinib. Sensitivity prediction was performed based on immunohistochemical staining according to method 1.

FIG. 6 shows the IC₅₀ values of PDAC-derived cell lines to Dasatinib and Sarcatinib. Sensitivity prediction was performed based on the gene-expression based method using the sensitivity predictor signature (method 2).

FIG. 7 gives an overview of the experimental outline of the in vivo drug sensitivity assays performed.

FIGS. 8-10 depict data from an in vivo drug test comparing the effects of Dasatinib either alone or in combination with Gemcitabine compared to untreated tumors. Dasatinib alone delayed tumor growth significantly in the tumors that were predicted to be sensitive. The combination of Dasatinib with Gemcitabine led to tumor regression in the sensitive PDAC while the combination had no significant better effect compared to Gemcitabine alone in the two other tumors.

FIG. 11 shows the FDR values for GSEA analysis on signatures predicting sensitivity to Src-inhibitors obtained by GSEA. GSEA was performed on expression profiles form the in vitro and xenograft PDAC models.

FIG. 12 shows the different sensitivities of the three PDAC subtypes described by Collisson et al. to Src-inhibitors in vitro. The cell lines were assigned to PDAC subtypes using the PDassigner as described (Collisson et al., loc. cit.). PDAC cells of the classical subtype have a significantly higher sensitivity (lower IC₅₀ values) than the other subtypes.

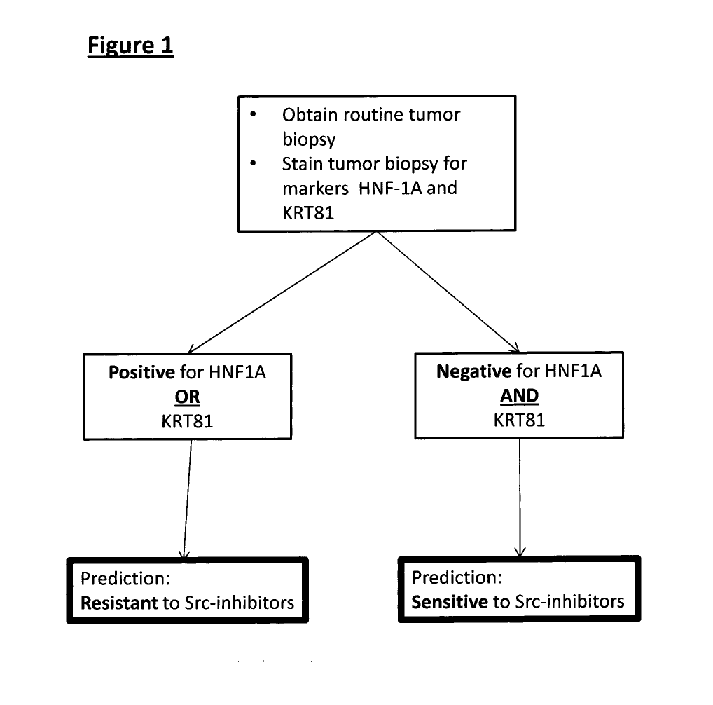

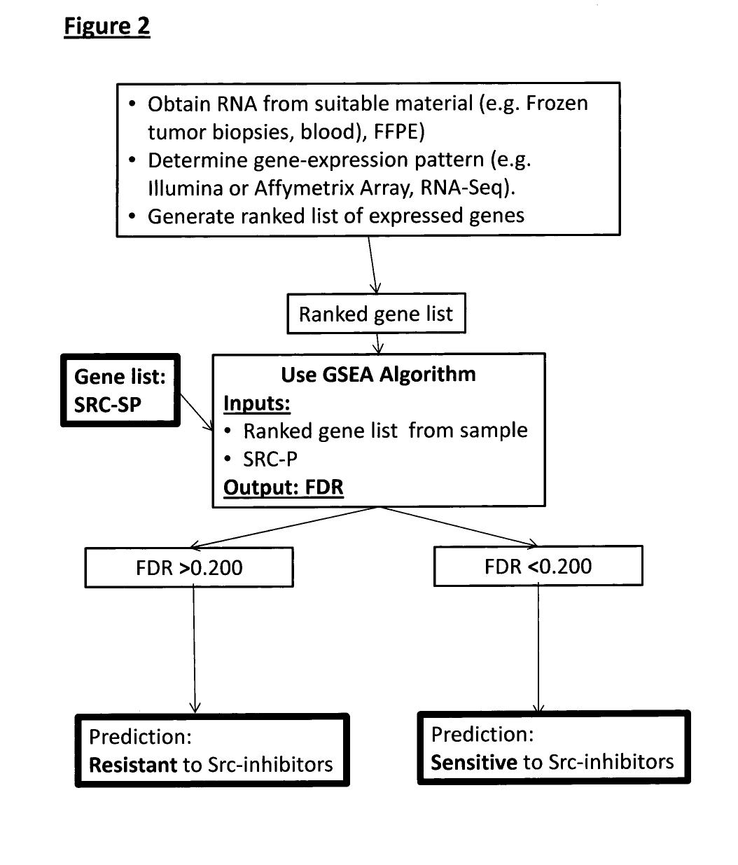

### DETAILED DESCRIPTION OF THE INVENTION

The present invention may be understood more readily by reference to the following detailed description of the invention and the examples included therein.

In one aspect, the present invention relates to a Src inhibitor for use in the treatment of (a) PDAC of the classical subtype and/or (b) a Src inhibitor sensitivity predictor-positive PDAC subtype.

In another aspect, the present invention relates to a method for the treatment of (a) PDAC of the classical subtype or (b) a Src inhibitor sensitivity predictor-positive PDAC subtype, comprising the step of administering a Src inhibitor to a patient in need thereof.

In the context of the present invention, “Src” relates to a protein (also called c-Src for “cellular Src”), which is a tyrosine kinase encoded by the proto-oncogene SRC, which is frequently overexpressed and highly activated in malignancies. Src is a member of a kinase family (the so-called “Src family”). Additional members of that family are: Lyn, Fyn, Lck, Hck, Fgr, Blk, Yrk and c-Yes.

In the context of the present invention, “PDAC” refers to pancreatic ductal adenocarcinoma, the most common type of pancreatic cancer, accounting for 95% of these tumors, arising within the exocrine component of the pancreas. It is typically characterized by moderately to poorly differentiated glandular structures on microscopic examination.

In the context of the present invention, “pancreatic cancer” refers to a cancer originating from transformed cells arising in tissues forming the pancreas.

In the context of the present invention, the term “classical PDAC”, refers to a PDAC subtype as identified by Collisson et al. (2011) based on its gene expression profile. In this study, a 62-gene panel was devised that enables classification of tumor samples into three subtypes, classical, quasi-mesenchymal and exocrine-like.

Tumors of the quasi-mesenchymal PDAC subtype give rise to poorly differentiated tumors characterized by abundant mitoses with abnormal spindle formation and cells with large cytoplasm and anisomorphic (i.e. of abnormal morphology) to pleomorphic (i.e. variable in shape) nuclei.

The classical or the exocrine-like PDAC subtype gives rise to tumors with a more differentiated growth pattern of medium-sized neoplastic duct-structures with only moderate variation in nuclear size and chromatin structure.

Using tumor models representing these three subtypes, it could be shown that keratin 81 and vimentin are markers for PDAC cells of the quasi-mesenchymal subtype, wherein (i) one or more transcription factors selected from HNF-1A, HNF-1B, FOXA2 (HNF3B), FOXA3 (HNF3G), HNF4G, and ONECUT1 (HNF6), (ii) one or more target genes regulated by HNF-1A, particularly the HNF-1A target genes listed in Table 2, and (iii) cadherin17 (CDH17), particularly HNF-1A and HNF-1B, are markers for cells of the exocrine-like subtype, whereas PDAC cells of the classical subtype are characterized by the absence of (i) keratin 81 and/or vimentin, and (ii) HNF-1A and/or HNF-1 B.

In a particular embodiment, PDAC of the classical subtype are identified by determining the absence of specific expression of the biomarkers keratin 81 and HNF-1A and/or HNF-1 B, particularly keratin 81 and HNF-1A.

In particular embodiments, PDAC of the classical subtype are identified by determining the absence of specific expression of one or more additional biomarkers selected from (i) transcription factors selected from HNF-1A, HNF-1B, FOXA2 (HNF3B), FOXA3 (HNF3G), HNF4G, and ONECUT1 (HNF6), (ii) one or more target genes regulated by HNF-1A, particularly the HNF-1A target genes listed in Table 2, and (iii) cadherin17 (CDH17).

In the context of the present invention, the term “specific expression” refers to the detection of a protein or a transcript in a sample compared to one or more comparator samples. The expression of an investigated marker is considered specific to a sample if of 500 analyzed tumor cells at least 1 tumor cell shows a signal above that observed with an unspecific control antibody and in the comparator sample or comparator samples no positive signal for the investigated marker can be detected.

In the context of the present invention, the term “specific expression” for analysis can also refer to the detection of the amount of a specific RNA-transcript in the total sample. The relative amount of the mRNA can be determined quantitatively (by e.g. qRT-PCR) by comparing it to one or more suited standards (e.g. the housekeeping genes beta-actin or GAPDH). Alternatively, the expression can be determined by comparing to other tumor samples or normal, non-cancerous, tissue. If the relative expression of the transcript is greater than a previously determined cutoff (e.g. 1.5-fold, 2-fold, 5-fold or 10-fold) the expression is considered to be specific for the sample.

In the context of the present invention, the term “absence” refers to the percentage of marker-positive tumor cells in a sample (determined as described in Section [0037]). If in 500 analyzed tumor cells of a sample no cell with a signal for the investigated marker is detected, the marker is considered to be ‘absent’ in the sample. Alternatively, if the level of a transcript (determined as described in Section [0038]) is below a previously determined cut-off, the transcript is considered to be ‘absent’ from the sample.

In the context of the present in invention, the term “Src inhibitor sensitivity predictor-positive PDAC subtype” refers to a subtype that is characterized by tumor cells scoring positive in a gene expression analysis using a ranked gene list as input for the GSEA-Algorithm (Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proc Natl Acad Sci USA 102, 15545-15550). In the context of the present invention, “scoring positive” means having a false discovery rate (“FDR”) of less than 0.200.

In a particular embodiment, the gene expression analysis is performed as described in Example 4 using the set of 30 genes shown in Table 1.

The present inventors have additionally found that cells of the classical PDAC subtype, and certain cells of the quasi-mesenchymal PDAC subtype, belong to a Src inhibitor sensitivity predictor-positive PDAC subtype, while the remaining cells of the quasi-mesenchymal PDAC subtype as well as cells of the exocrine-like subtype are Src-inhibitor resistant (Src inhibitor sensitivity predictor-negative PDAC subtype).

In particular embodiments, the present invention relates to a Src inhibitor for use in the treatment of PDAC of the classical subtype.

In particular embodiments, the present invention relates to a Src inhibitor for use in the treatment of a Src inhibitor sensitivity predictor-positive PDAC subtype.

In particular embodiments, the Src inhibitor is selected from Dasatinib (BMS-354825), Bosutinib (SKI-606), and Saracatinib (AZD0530), particularly Dasatinib.

In another aspect, the present invention relates to a Src inhibitor for use in the treatment of (a) PDAC of the classical subtype and/or (b) a Src inhibitor sensitivity predictor-positive PDAC subtype, in combination with a second chemotherapeutic agent.

In another aspect, the present invention relates to a method for the combination treatment of (a) PDAC of the classical subtype or (b) a Src inhibitor sensitivity predictor-positive PDAC subtype, comprising the step of administering a Src inhibitor in combination with a second therapeutic agent to a patient in need thereof.

In certain embodiments, the second therapeutic agent is a chemotherapeutic agent. In particular embodiments, the chemotherapeutic agent is selected from Gemcitabine, Fluorouracil (5-FU or f5U) and derivatives thereof, and a platinum compound, particularly Oxaliplatin. In particular embodiments, the chemotherapeutic agent is Gemcitabine.

In particular embodiments, the PDAC is resectable. In particular such embodiments, the Src inhibitor, and optionally, the second chemotherapeutic agent, is/are administered after operative removal of the primary carcinoma.

### EXAMPLES

### Example 1

### Assessment of PDAC Subtype-Specific Gene Set Enrichments and Vulnerabilities

In order to identify the PDAC subtype and subtype-specific pathway enrichments that could be exploited therapeutically, gene set enrichment analysis was used first to identify the subtype by using the 62-Gene PDAssigner as described (Collisson et al., loc. cit.). Gene-expression profiles of stable PDAC cell lines were then compared against the Broad Institute's MSig database of >6700 gene sets. Two publicly available datasets of RNA expression data obtained from more than 70 primary PDAC tumor samples were included in the analysis. Total RNA was isolated from the stable PDAC cell lines using the miRNAeasy kit (Qiagen, Hilden). Gene expression analysis was performed using the Illumina BeadChip Technology (HumanHT-12). Gene set enrichment analysis on normalized data was conducted as described previously (Subramanian et al., loc. cit.).

Signatures derived from the stable PDAC cell lines were more sensitive and robust in detecting relevant gene sets compared to the two publicly available datasets used as indicated by lower FDR rates and the presence of signatures in only one of the public datasets. The analysis revealed a gene set predicting sensitivity to the Src/Bcr-Abl inhibitor Dasatinib, which was enriched in the classical subtype, while conversely a higher resistance was predicted in the other two subtypes.

### Example 2

### In Vitro Drug Screens

An in vitro screen to uncover subtype-specific drugs was carried out based on the predicted subtype-specific pathway-dependencies and drug sensitivities. A small-scale inhibitory screen with compounds selected to target pathways identified by gene-expression analysis was compiled. The selected compounds were tested on all stable PDAC cell lines. 8,000 cells/well in a 96-well plate were incubated with 10 μM of the compounds. Cell growth was determined after 72 h using Cell Titer Blue (Promega, Mannheim). Raw measurements were converted to Z-values and percent growth inhibition was calculated using positive and negative controls distributed evenly throughout the plate.

Gemcitabine was obtained from Sigma. All remaining compounds were obtained from Enzo Life Sciences (Farmingdale). Stock concentrations of 100 mM were prepared in water-free DMSO, Gemcitabine was dissolved at 1 mM in sterile buffered saline.

The screen confirmed the predicted sensitivity of the classical subtype to Dasatinib. To obtain more precise drug sensitivity profiles, the stable PDAC cell lines were subjected to dose-escalation studies using these targeted agents. For calculation of the IC₅₀, 3-fold serial dilutions of selected compounds were screened in quadruplicates, incubated for 72 h after which cell viability was assessed using CellTiterBlue as described. Raw data was normalized to positive and negative controls present on each individual plate IC₅₀ values were calculated using GraphPad Prism (Graph Pad Software, La Jolla).

The differential sensitivities were confirmed in vitro. Both the exocrine-like and quasi-mesenchymal PDAC cells were relatively resistant to Dasatinib, whereas the classical subtype showed a >10,000 fold lower IC₅₀ value, which is within the therapeutically relevant range.

### Example 3

### Determination of IC₅₀ Values In Vitro

For calculation of the IC₅₀ serial dilutions of selected compounds were screened in quadruplicates. In brief, 8,000 cells/well were seeded 24 h prior to addition of individual compounds in 96-well plates. After incubation for 72 h, cell viability was assessed using CellTiterBlue (Promega, Mannheim) as described. Raw data was normalized to positive and negative controls present on each individual plate. IC₅₀ values were calculated using GraphPad Prism (Graph Pad Software, La Jolla). Dasatinib and Saracatinib were from LC Laboratories, (Woburn). Stock concentrations of 100 mM were prepared in water-free DMSO.

### Example 4

### Prediction of Src-Inhibitor Sensitivity Using mRNA Expression

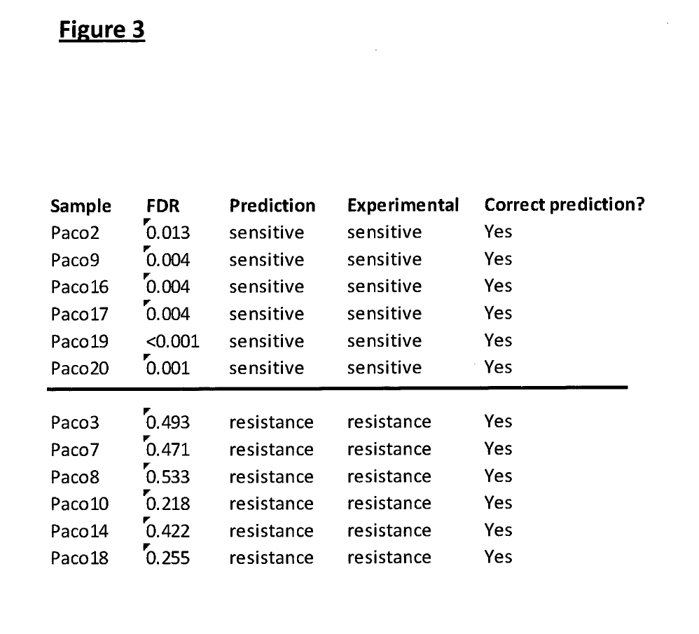

Total RNA was isolated from PDAC cell lines or tumor xenografts using the miRNAeasy kit (Qiagen, Hilden). Gene expression analysis was performed with the Illumina BeadChip Technology (HumanHT-12). The resulting list of genes with their associated signal intensities was normalized by quantile normalization and used as input for the GSEA algorithm (Subramanian et al., loc. cit.). While normalization increases the sensitivity and specificity of the method, the GSEA can also be performed successfully without previous normalization. Each list was compared separately against the sensitivity predictor signature (SRC-SP) described in FIG. 3, and the FDR calculated. A FDR cutoff value of 0.200 was determined to be optimal for accurate classification of samples into either Src-inhibitor sensitive or resistant. Samples resulting in a FDR<0.200 are predicted to be sensitive, while a FDR>0.200 predicts resistance.

### Example 5

### In Vivo Drug Screens

The differential response of tumors to Dasatinib was further explored in a subtype-specific mouse model of human PDAC. Three groups of mice that each carried transplanted human tumors of one either the sensitive or the resistant subtypes were treated with Dasatinib, either alone or in combination with the chemotherapeutic agent Gemcitabine.

PACO tumors were established by injecting 5×10⁵ cells subcutaneously into a cohort 20 NOD.Cg-Prkdcscid Il2rgtm1Wjl (NSG) mice. After the tumors reached a size of approx. 200 mm³, mice were randomized into two groups of each 10 mice—Control, Gemcitabine, Dasatinib or combination of both. Gemcitabine was dissolved in 0.8% buffered saline and administered twice weekly at 125 mg/kg i.p. Dasatinib was prepared in citrate/citric acid buffer (pH 3) and administered daily via oral gavage at 25 mg/kg. Tumor volume was determined twice weekly via caliper measurements and calculated according the formula (length×height×width)×(π/6). Relative tumor growth was calculated for each individual tumor in relation to the volume calculated as of the start of the experiment. All animal care and procedures followed German legal regulations and were previously approved by the governmental review board of the state of Baden-Wuerttemberg, Germany.

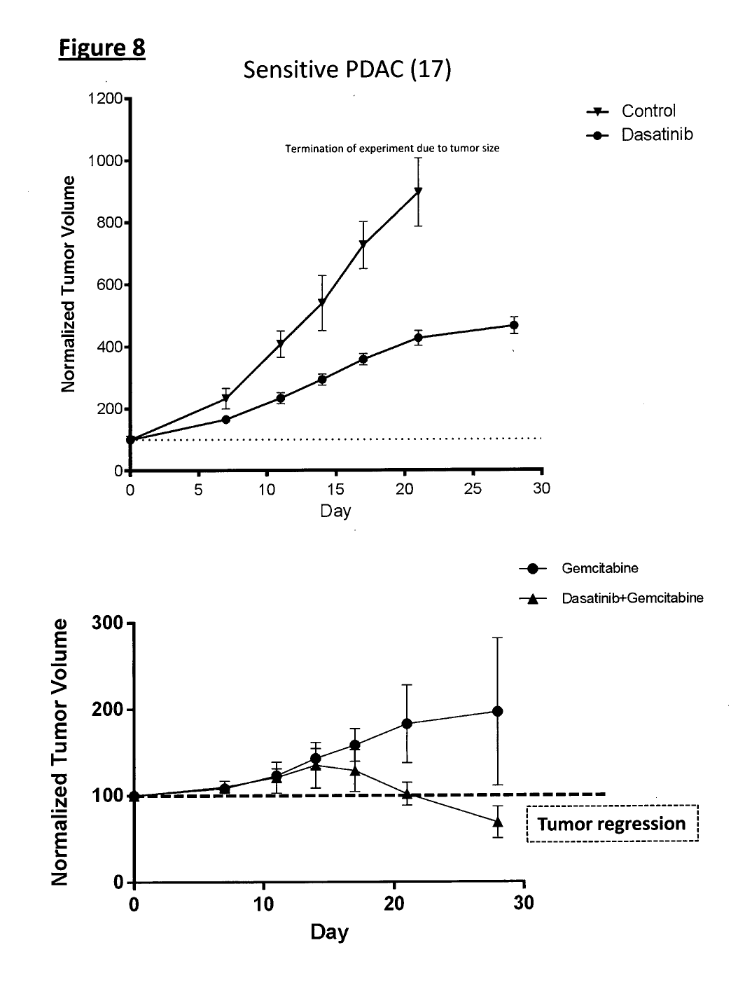

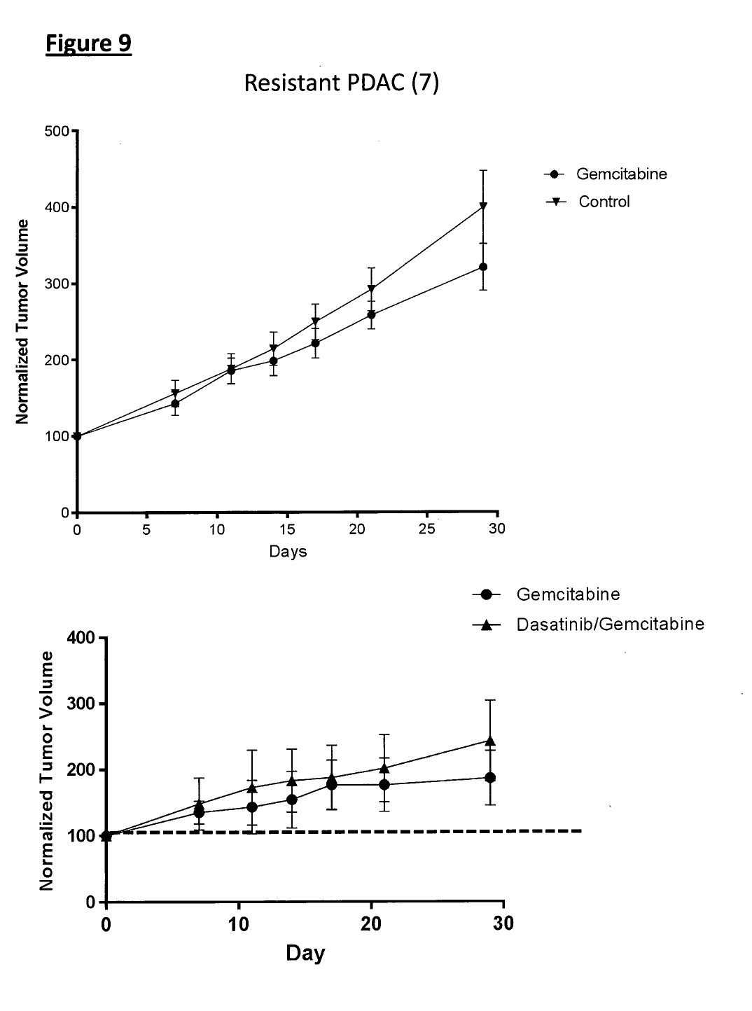

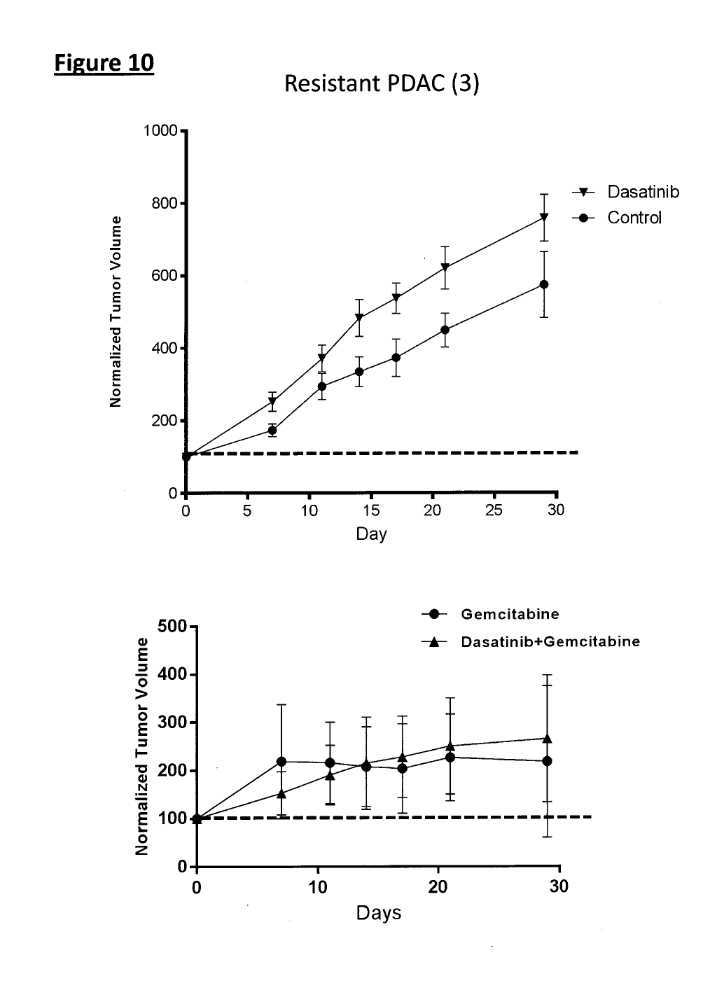

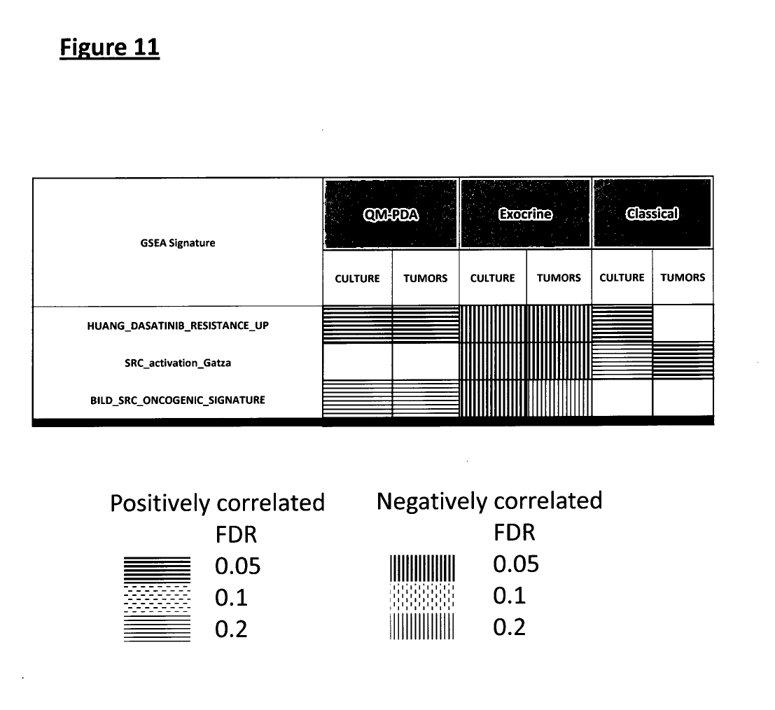

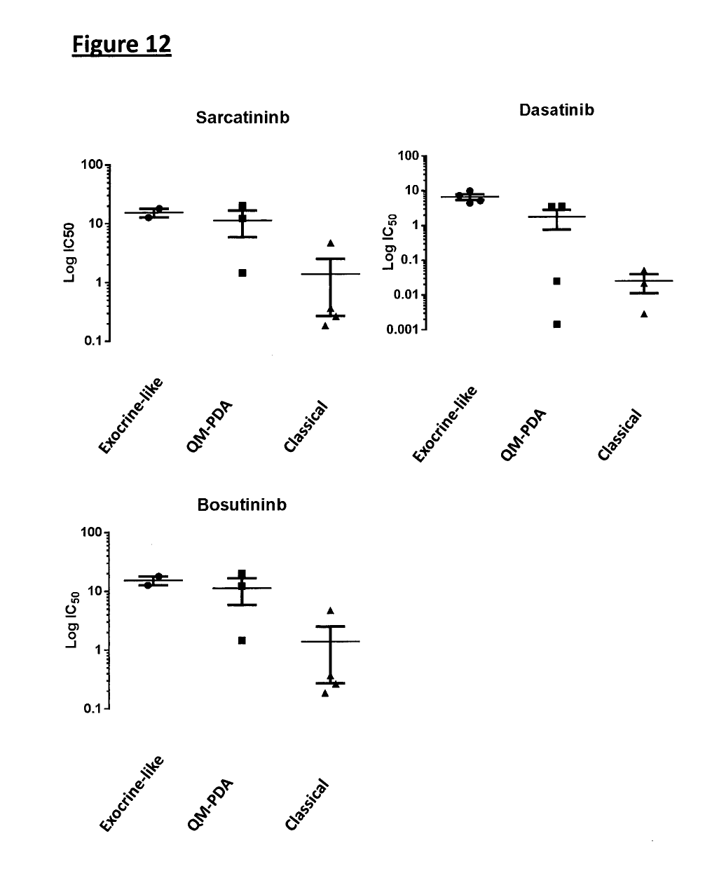

An overview of the experimental scheme is shown in FIGS. 8-10. Tumor size is expressed as relative volume normalized to the day of start of treatment. As demonstrated, Dasatinib when used alone had a significant growth-inhibitory effect only on the tumors of the classical subtype. Gemcitabine alone showed a growth-inhibitory effect on tumors of all three subtypes, however did not lead to a complete stop in tumor growth. When Dasatinib was used in combination with Gemcitabine, a decrease in tumor size compared to the original was observed only in the tumors of the classical subtype while the combination had no greater effect than gemcitabine alone or even showed an increased tumor growth in the exocrine-like subtype.

### Example 6

### Determination of Tumor Subtype by Immunohistochemistry

For formalin-fixed and paraffin-embedded tissue samples, sections of 3 μm-5 μm were de-paraffinized and rehydrated. Antigens were retrieved by boiling in a steam pot at pH 6 (Dako target retrieval solution, Dako, Glostrup) for 15 min, allowed to cool for 30 min and washed in distilled water. Nonspecific binding was blocked using the Linaris Avidin/Biotin blocking Kit (Vector Labs, Burlingame) according to the manufacturer's instructions. Slides were incubated with primary antibodies for 30 min, rinsed in PBS-T (PBS with 0.5% Tween-20), incubated for 20 min with the appropriate secondary antibody using the Dako REAL Detection System and rinsed in PBS-T. After blocking of endogenous peroxidase and incubation with Streptavidin HRP (20 min at RT), slides were developed with AEC (Dako) and counterstained with Hematoxylin. The staining is performed on two separate sections or two separate areas on a section for HNF-1 and keratin 81, respectively.

Evaluation of Staining:

Only tumor cells are considered in the evaluation of the staining for subtype assignment. 500 tumor cells are evaluated in each specimen. A signal is considered positive if the observed signal can be clearly distinguished from the background staining observed with an isotype control antibody on a comparable specimen. The specimen is considered positive for Keratin 81, if at least one tumor cell shows a clearly detectable intracellular signal. The specimen is considered positive for HNF-1 if at least one of the tumor cells shows a clearly detectable nuclear staining. The subtype is then determined as follows:

Keratin 81 positive/HNF-1 negative: quasi-mesenchymal subtype
Keratin 81 negative/HNF-1 positive: exocrine-like subtype
Keratin 81 negative/HNF-1 negative: classical subtype
Keratin 81 positive/HNF-1 positive: undetermined

### Example 7

### Correlation of Marker Expression with Patient Survival in a Tissue Microarray

Stratification of patients combined with subtype-specific therapeutic approaches is becoming increasingly important and has already been shown to improve the efficacy of treatments in several types of cancer. However, until now attempts to stratify PDAC patients into clinically meaningful groups have yielded mixed results (Stathis, A., and Moore, M. J. (2010). Advanced pancreatic carcinoma: current treatment and future challenges. Nat Rev Clin Oncol 7, 163-172). This could in part be attributed to the presence of at least three subtypes, complicating single-marker approaches. With a set of two markers we can unambiguously identify all three PDAC subtypes in the PACO model. Application of those markers to a cohort of 258 PDAC patients confirmed a non-overlapping staining; suggesting the robustness of our two marker-set when applied to patients groups. This allowed us to stratify a large cohort of patients into three groups, HNF1A/B positive (exocrine-like), keratin 81 (KRT81) positive (QM-PDA) and double-negative (classical). We found a significant difference of overall survival between the three groups. KRT81-positive patients had the worst prognosis (mean OS=16.5 months), while HNF-1A/B positive patients had the best outcome (mean OS=43.5 months) and the double negative were intermediate (mean OS=26.3 months). Our markers, thus for the first time allow for a clinically meaningful stratification of PDAC patients and can be easily incorporated into routine diagnosis providing the possibility for stratification of PDAC patients to guide current and novel treatments.

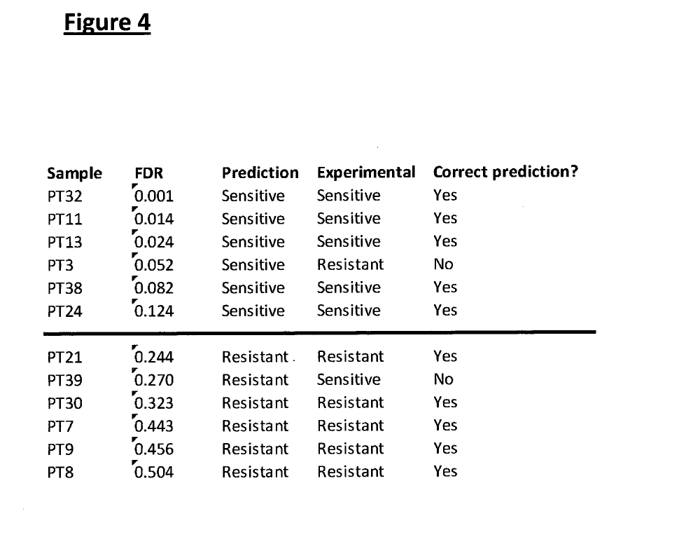

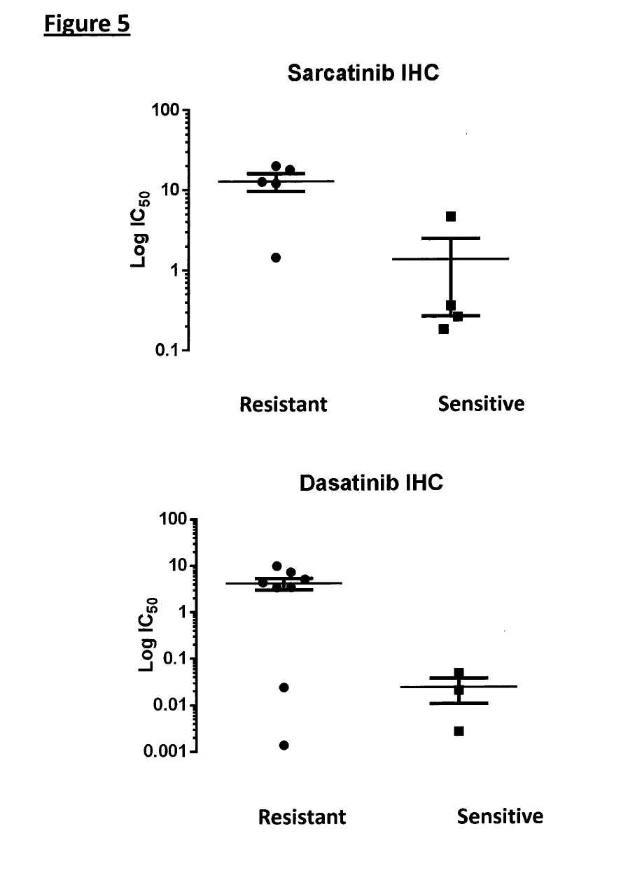

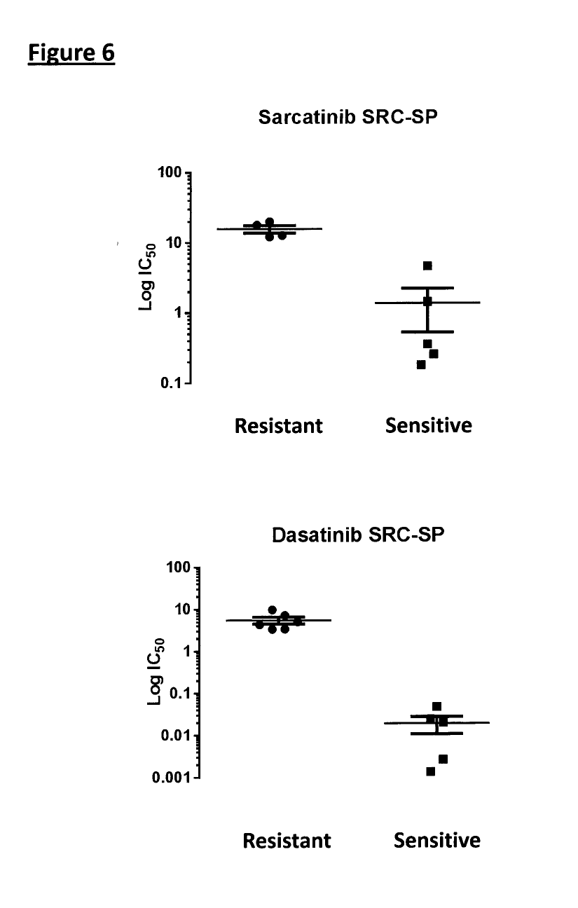

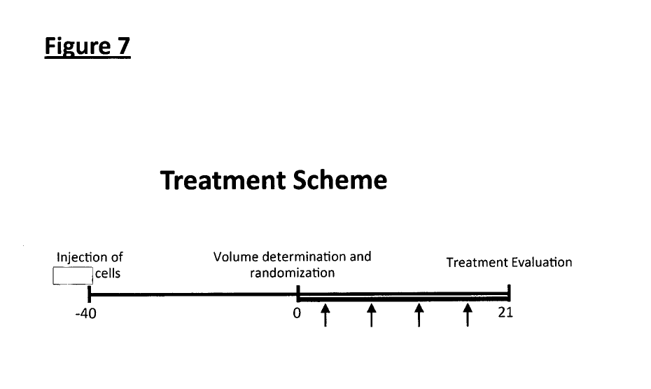

The tissue microarray was constructed from patients that received partial pancreatoduodenectomy for PDAC between 1991 and 2006 at the Charite University Hospital Berlin. The use of this tumor cohort for biomarker analysis has been approved by the Charite University ethics committee (EA1/06/2004). Patient characteristics are summarized in FIG. 4.

Formalin-fixed and paraffin-embedded tissue samples were used to generate tissue microarrays as described previously (Weichert, W., Roske, A., Gekeler, V., Beckers, T., Ebert, M. P., Pross, M., Dietel, M., Denkert, C., and Rocken, C. (2008). Association of patterns of class I histone deacetylase expression with patient prognosis in gastric cancer: a retrospective analysis. The lancet oncology 9, 139-148). Briefly, three morphologically representative regions of the paraffin ‘donor’ blocks were chosen. Three tissue cylinders of 0.6 mm diameter representing these areas were punched from each sample and precisely arrayed into a new ‘recipient’ paraffin block using a customer built instrument (Beecher Instruments, Silver Spring, Md., USA).

TABLE 1
List of genes contained in the SRC-SP predictor. Table 1 shows the 30 genes used in the Src inhibitor sensitivity predictor SRC-SP (see
Example 4). The gene list was compiled from gene-expression patterns derived from novel PDAC cell lines. The 30 genes that showed the
highest differential expression between Src inhibitor sensitive and resistant PDAC cell lines were included in the classifier.

| Gene-Symbol | Refseq ID | Definition |
|---|---|---|
| IFI44L | NM_006820.1 | Homo sapiens interferon-induced protein 44-like (IFI44L), mRNA. |
| IFI6 | NM_022872.2 | Homo sapiens interferon, alpha-inducible protein 6 (IFI6), transcript variant 2, mRNA. |
| MX1 | NM_002462.2 | Homo sapiens myxovirus (influenza virus) resistance 1, interferon-inducible protein p78 (mouse) |
| | | (MX1), mRNA. |
| OAS2 | NM_016817.2 | Homo sapiens 2′-5′-oligoadenylate synthetase 2, 69/71 kDa (OAS2), transcript variant 1, mRNA. |
| RARRES3 | NM_004585.3 | Homo sapiens retinoic acid receptor responder (tazarotene induced) 3 (RARRES3), mRNA. |
| IFI44 | NM_006417.3 | Homo sapiens interferon-induced protein 44 (IFI44), mRNA. |
| IFIT1 | NM_001548.3 | Homo sapiens interferon-induced protein with tetratricopeptide repeats 1 (IFIT1), transcript variant 2, |
| | | mRNA. |
| IFIT3 | NM_001031683.1 | Homo sapiens interferon-induced protein with tetratricopeptide repeats 3 (IFIT3), mRNA. |
| IFIT3 | NM_001549.2 | Homo sapiens interferon-induced protein with tetratricopeptide repeats 3 (IFIT3), mRNA. |
| IFI6 | NM_022873.2 | Homo sapiens interferon, alpha-inducible protein 6 (IFI6), transcript variant 3, mRNA. |
| ISG15 | NM_005101.1 | Homo sapiens ISG15 ubiquitin-like modifier (ISG15), mRNA. |
| IFIT2 | NM_001547.4 | Homo sapiens interferon-induced protein with tetratricopeptide repeats 2 (IFIT2), mRNA. |
| OASL | NM_198213.1 | Homo sapiens 2′-5′-oligoadenylate synthetase-like (OASL), transcript variant 2, mRNA. |
| IFI27 | NM_005532.3 | Homo sapiens interferon, alpha-inducible protein 27 (IFI27), transcript variant 2, mRNA. |
| EPSTI1 | NM_033255.2 | Homo sapiens epithelial stromal interaction 1 (breast) (EPSTI1), transcript variant 2, mRNA. |
| APOBEC3G | NM_021822.1 | Homo sapiens apolipoprotein B mRNA editing enzyme, catalytic polypeptide-like 3G (APOBEC3G), |
| | | mRNA. |
| HERC5 | NM_016323.2 | Homo sapiens hect domain and RLD 5 (HERC5), mRNA. |
| BST2 | NM_004335.2 | Homo sapiens bone marrow stromal cell antigen 2 (BST2), mRNA. |
| OAS3 | NM_006187.2 | Homo sapiens 2′-5′-oligoadenylate synthetase 3, 100 kDa (OAS3), mRNA. |
| APOBEC3G | NM_021822.1 | Homo sapiens apolipoprotein B mRNA editing enzyme, catalytic polypeptide-like 3G (APOBEC3G), |
| | | mRNA. |
| CXCL10 | NM_001565.2 | Homo sapiens chemokine (C-X-C motif) ligand 10 (CXCL10), mRNA. |
| XAF1 | NM_199139.1 | Homo sapiens XIAP associated factor 1 (XAF1), transcript variant 2, mRNA. |
| HOXB2 | NM_002145.3 | Homo sapiens homeobox B2 (HOXB2), mRNA. |
| UBE2L6 | NM_004223.3 | Homo sapiens ubiquitin-conjugating enzyme E2L 6 (UBE2L6), transcript variant 1, mRNA. |
| RSAD2 | NM_080657.4 | Homo sapiens radical S-adenosyl methionine domain containing 2 (RSAD2), mRNA. |
| CDKN2B | NM_078487.2 | Homo sapiens cyclin-dependent kinase inhibitor 2B (p15, inhibits CDK4) (CDKN2B), transcript variant |
| | | 2, mRNA. |
| IRF7 | NM_004029.2 | Homo sapiens interferon regulatory factor 7 (IRF7), transcript variant b, mRNA. |
| ITGB2 | NM_000211.1 | Homo sapiens integrin, beta 2 (antigen CD18 (p95), lymphocyte function-associated antigen 1; |
| | | macrophage antigen 1 (mac-1) beta subunit) (ITGB2), mRNA. |
| SAMD9L | NM_152703.2 | Homo sapiens sterile alpha motif domain containing 9-like (SAMD9L), mRNA. |
| ERAP2 | NM_022350.2 | Homo sapiens endoplasmic reticulum aminopeptidase 2 (ERAP2), mRNA. |

TABLE 2
List of HNF-1 target genes that are
expressed in Exocrine-like PDAC

| | | |
|---|---|---|
| SLC5A1 | MSX2 | TINAG |
| FGFR4 | MMP11 | USP3 |
| ANXA13 | TLE4 | HABP2 |
| C11ORF9 | SFRS7 | CREB5 |
| TM4SF4 | SGK2 | CLDN10 |
| KIF12 | GUCA2A | PURA |
| RNASE4 | SERPINA4 | PRODH2 |
| UGT1A1 | ALB | GRB7 |
| TCEA3 | GJB1 | AGT |
| CDH17 | SLC37A4 | TTR |
| SLC4A4 | SPINK1 | SERPING1 |
| NR5A2 | SLC12A2 | MLL |
| LGALS2 | TBL1X | THAP11 |
| NR1H4 | SLC1A1 | |
| UGT1A6 | RBP4 | |
| GATA6 | ARHGAP12 | |
| NPAS2 | NDST2 | |
| NFE2L2 | EML4 | |
| FAM20C | NEO1 | |
| TBXAS1 | THRA | |
| IGFBP1 | PRDM1 | |
| CDAN1 | LPP | |
| GUCA2B | PLS3 | |
| SLC3A1 | YES1 | |
| FOXA2 | AFM | |
| STAT5B | C14ORF138 | |
| MSH5 | BACE2 | |
| APOM | UBE3A | |
| FXYD2 | LRRC19 | |
| HPN | AQP4 | |
| SEMA4G | LRRFIP2 | |
| ROBO3 | BPHL | |
| SLC39A14 | C5 | |
| SLC7A9 | CYFIP2 | |
| NFIX | SLC39A5 | |
| PDGFRA | AXIN2 | |
| RAB3IP | TMEM27 | |
| GC | SULF2 | |
| ANPEP | MIA2 | |
| CRB3 | STAG2 | |

We claim:

1. A Src inhibitor for use in the treatment of PDAC of the classical subtype and/or of a Src inhibitor sensitivity predictor-positive PDAC subtype.

2. A method for the treatment of PDAC of the classical subtype or of a Src inhibitor sensitivity predictor-positive PDAC subtype comprising the step of administering a Src inhibitor to a patient in need thereof.

3. A Src inhibitor for use in the treatment of (a) PDAC of the classical subtype and/or (b) a Src inhibitor sensitivity predictor-positive PDAC subtype, in combination with a second chemotherapeutic agent.

4. A method for the combination treatment of (a) PDAC of the classical subtype or (b) a Src inhibitor sensitivity predictor-positive PDAC subtype, comprising the step of administering a Src inhibitor in combination with a second therapeutic agent to a patient in need thereof.

5. The Src inhibitor of claim 3, wherein the second therapeutic agent is a chemotherapeutic agent, particularly a chemotherapeutic agent selected from Gemcitabine, Fluorouracil and derivatives thereof, and a platinum compound, particularly Oxaliplatin.

6. The Src inhibitor of claim 1, wherein the treatment is the treatment of PDAC of the classical subtype.

7. The Src inhibitor of claim 1, wherein the treatment is the treatment of a Src inhibitor sensitivity predictor-positive PDAC subtype.

8. The Src inhibitor of claim 1, wherein the Src inhibitor is selected from Dasatinib, Bosutinib, and Saracatinib, particularly Dasatinib.
